(19) United States
(12) Patent Application Publication
(10) Pub. No.: US 2023/0005153 A1
(43) Pub. Date: Jan. 5, 2023

(54) QUANTIFICATION AND VISUALIZATION OF MYOCARDIUM FIBROSIS OF HUMAN HEART

(71) Applicant: Arbind Kumar GUPTA, Bengaluru (IN)

(72) Inventor: Arbind Kumar GUPTA, Bengaluru (IN)

(21) Appl. No.: 17/779,884

(22) PCT Filed: Dec. 4, 2020
(86) PCT No.: PCT/IN2020/051003

§ 371 Date: May 25, 2022

(30) Foreign Application Priority Data
Dec. 5, 2019 (IN) 201941050089

(51) Int. Cl. G06T 7/11; A61B 5/00; G06T 7/168

(52) U.S. Cl. CPC G06T 7/11; A61B 5/7425; A61B 5/4842; G06T 7/168; G06T 2207/10081; G06T 2207/10088; G06T 2207/10116; G06T 2207/10104; G06T 2207/10108; G06T 2207/10132; G06T 2207/30048

(57) ABSTRACT

Embodiments of the present disclosure are related to providing a method and device processing a first set of volumetric image data comprising cross-sectional images of a myocardium and displaying a second set of volumetric image data of the myocardium. A curved plane to rectangular plane transformation of cross-sectional images of myocardium of human heart is proposed. After the transformation, a combined and reconstructed set of myocardium images are superimposed with a modified Bull's Eye View (BEV) map and corresponding parameters indicating extent of fibrosis to obtain a second set of volumetric image data of myocardium. In addition to quantifying and displaying the extent of fibrosis, the proposed solution preserves neighborhood and adjacency criteria of abnormal tissues of myocardium walls of human heart.

### TECHNICAL FIELD

The subject matter relates to image processing of medical images. More specifically, it relates to visualization of the extent of myocardium fibrosis of human heart for better characterization of the fibrosis.

### PRIORITY INFORMATION

The subject matter takes priority from the provisional application of application number: 201941050089, TITLE: “CPR _{2D} Reconstruction” and filed in Indian Patent Office, Chennai Branch on 05 Dec. 2019.

### BACKGROUND

One of the serious health problems faced by human beings world over is heart failure. While there are several causes and factors leading to heart failures, one of the leading causes is Ischemic Cardio Myopathy (ICM). ICM is a term that refers to the heart's decreased ability to pump blood, due to myocardial damage brought upon by ischemia. Ischemia is defined as inadequate blood supply (circulation) to a local area due to blockage of the blood vessels supplying the area. In case of ischemia of heart, thickening of heart muscles known as fibrosis of myocardium occurs. Fibrosis of myocardium is a subject of intense study by Cardiologists world over in order to advice preventive measures to affected people.

Images of Myocardium from diagnostic imaging systems known as ‘imaging modalities’, such as Magnetic Resonance Imaging (MRI)are found to be useful for studying the extent of fibrosis. The images obtained from such diagnostic systems e.g., Late Gadolinium Enhanced MRI (LGE-MRI), are captured in the form of layers or slices of cross-sectional images of heart muscles. These set of 2D images have to be transformed, reconstructed and presented to Cardiologists by means of advanced image processing techniques for unambiguous visualization of abnormalities of heart muscles. Quantification, localization and visualization of fibrosis of myocardium are routinely and repeatedly performed by Cardiologists. Hence a user-friendly and efficient method of visualization may be of immense help to a Specialist Doctor in his/her clinical decision-making process to assess and advice about the extent of cardiomyopathy and its viability, i.e. restoration of normal cardiac function.

The existing approach for visualization of fibrosis is done using Bull's Eye View (BEV) graph of fibrosis of myocardium of human heart. BEV scheme of dividing the myocardial wall in to 17 segments is based on the arteries supplying blood to the myocardium. In the BEV graph, the myocardium is divided into a graphical representation of three ring-like structures which are further divided into 17 segments arranged in a circular order as explained in this document subsequently in the description of FIG. 2a and FIG. 2b. BEV map and BEV graph refer to the same type of presentations in this document. In this document, by fibrosis, it is referred to myocardium fibrosis of human heart. The existing system of representation and study of fibrosis by means of a BEV map presents many shortcomings to the Physicians studying the fibrosis of epicardium of myocardium for characterization and further necessary action. It is not possible to see transmurality of fibrosis in BEV. Also, information about the spatial distribution of fibrosis on the entire surface of myocardium is absent in this scheme. Further, correlation of fibrosis with wall motion and thickness analysis and abnormality analysis are not possible in the present BEV map of fibrosis. Dynamic behaviour of heart from the time series images is studied by adopting a cumbersome procedure in the present representation and analysis of myocardium fibrosis.

### SUMMARY

It is therefore one of the objects of the subject matter to provide a method and device that overcomes the above and other limitations. The subject matter provides a method and device that meets the above and other challenges. The proposed solution is based on a transformation, reconstruction, and superimposition of segment boundaries, as per BEV definition, on the reconstructed myocardium image of a human heart for efficient visualization. Further, a set of parameters indicative of extent of fibrosis of the reconstructed myocardium is superimposed on the transformed myocardium with segment boundaries and displayed for efficient interpretation and characterization of myocardium fibrosis. The proposed solution offers a better method of visualization, interpretation and quantification of fibrosis of myocardium.

According to a first aspect, the present subject matter provides a method for processing a first set of volumetric image data comprising cross-sectional images of a myocardium and displaying a second set of volumetric image data of the myocardium, the method comprising: pre-processing the first set of volumetric image data received from an image capturing system to obtain a segmented set of myocardium images and their respective centroids and radii, transforming each one of the segmented myocardium images from a polar coordinate system into a rectangular coordinate system using their respective centroids and radii to obtain a transformed set of myocardium images that maintains pixel size (and aspect ratio) for different imaging planes, combining the transformed set of myocardium images to obtain a reconstructed set of myocardium images, demarcating a plurality of image segments conforming to a Bull's Eye View (BEV) map on the reconstructed set of myocardium images, estimating a set of parameters indicative of extent of fibrosis of the myocardium from the respective pixel values of each one of the demarcated plurality of BEV segments, superimposing the estimated sets of parameters indicative of extent of fibrosis of the myocardium at their respective positions on the plurality of BEV image segments to obtain the second set of volumetric image data of the myocardium and displaying the second set of volumetric image data of the myocardium on a display device.

In a second embodiment, the estimated set of parameters indicative of the extent of fibrosis are selected from a group consisting essentially of average fibrosis, maximum fibrosis, wall thickness and wall displacement of the myocardium.

According to a third embodiment, the method further comprises a step of navigating in radial direction to display respective cross-sectional views as a 2-dimensional image derived from the second set of volumetric image data of the myocardium. For each of these 2-dimensional images for different radial distance, the corresponding fibrosis regions are shown, thereby allowing a user to see transmurality of fibrosis.

According to a fourth embodiment, at least one of the estimated set of parameters is displayed on each one of the derived 2-dimensional image of the second set of volumetric image data of the myocardium.

According to a fifth embodiment, the first volumetric image of the myocardium is received from at least one of the CT, MRI, ultrasound, X-ray, PET, and SPECT system representations of the myocardium.

According to a sixth embodiment, the method further comprises steps of attaching a pre-decided percentage from the right side of the reconstructed set of myocardium images to the left side of the reconstructed set of myocardium images, and attaching a pre-decided percentage from the left side of the reconstructed set of myocardium images to the right side of the reconstructed set of myocardium images, before demarcating the plurality of image segments conforming to the Bull's Eye View (BEV) map, thereby providing an augmented view of the reconstructed set of myocardium images.

According to a seventh embodiment the step of displaying further comprises receiving and displaying at least one of: a short axis image set (images taken at different time instants covering one full cycle of heart beat), a 2-Chamber image set, a 3-Chamber image set and a 4-Chamber image set from the image capturing system in a plurality of display windows in addition to the second set of volumetric image data of the myocardium. The 2-chamber, 3-chamber and 4-chamber image sets are taken along long axis of left ventricle such that 2 chambers, 3 chambers and 4 chambers of heart is visible in the image respectively.

According to an eighth embodiment, the step of displaying further comprises receiving and displaying at least one of : the short axis image set, the 2-Chamber image set, a 3-Chamber image set and a 4-Chamber image set as a sequence of a plurality of images in a predetermined time sequence, thereby displaying the beating of a heart in the display device.

According to a ninth embodiment, the step of displaying further comprises: displaying a plane of the short axis image set corresponding to a user defined line on the second set of volumetric image data and updating the displayed image with the corresponding plane of the short axis image set corresponding to the shifting of the user defined line on the second set of volumetric image data.

According to a tenth embodiment the step of displaying further comprises wherein the step of displaying further comprises displaying on a display window at least one of the 2-Chamber image, 3-Chamber image and 4-Chamber image interactively chosen from the respective set of images.

According to a second aspect, the present subject matter provides a device configured to perform processing of a first set of volumetric image data comprising cross-sectional images of a myocardium comprising pixel values and displaying a second set of volumetric image data of the myocardium, the device comprising a processing element adapted to carry out the steps of: pre-processing the first set of volumetric image of the myocardium received from an image capturing system to obtain a segmented set of myocardium images and respective centroids and radii, transforming each one of the segmented myocardium images from a polar coordinate system into a rectangular coordinate system using the respective centroids and radii to obtain a transformed set of myocardium images that maintains pixel size (and aspect ratio) for different short axis imaging planes, combining the set of transformed set of myocardium images to obtain a set of reconstructed set of myocardium images, demarcating a plurality of segment boundaries conforming to a Bull's Eye View (BEV) map on the reconstructed set of myocardium images, estimating a set of parameters indicative of extent of fibrosis of the myocardium from the respective pixel values of each one of demarcated plurality of BEV image segments, superimposing the estimated sets of parameters indicative of extent of fibrosis of the myocardium at their respective positions on the plurality of BEV image segments to obtain the second set of volumetric image data image and displaying the second set of volumetric image data of the myocardium on a display device.

### BRIEF DESCRIPTION OF DRAWINGS

The subject matter is now described with reference to the accompanying figures, in that:

FIG. 1a shows a schematic representation of a cross-section of a myocardium and related constituents of a human heart;

FIG. 1b shows a schematic diagram of imaging planes and axes of imaging of a human heart;

FIG. 1c shows a schematic representation of three sections perpendicular to the long axis of a human heart;

FIG. 2a shows a schematic representation of further sub-division of the three sections shown in FIG. 1c into 17 segments of myocardium;

FIG. 2b shows a representation of a Bull's Eye View (BEV) map of representation of the 17 segments of myocardium;

FIG. 3 shows a schematic diagram of a method 300 according to one embodiment of the present subject matter;

FIG. 4 shows a schematic diagram of a step 370 of the method 300 according to one embodiment of the present subject matter;

FIG. 5a shows a representation of an actual slice of cross-sectional image of a myocardium in a MRI image;

FIG. 5b shows a of a cross-sectional image of the myocardium wherein a detection mask is superimposed on the cross sectional image of the myocardium;

FIG. 5c shows the segmented part of the cross sectional view of the myocardium;

FIG. 6a shows a schematic representation of myocardium in an image for a given slice position z;

FIG. 6b shows a representation of the transformed myocardium image for the slice position z;

FIG. 6c shows a representation of three image slices assembled in a three dimensional frame;

FIG. 7 shows an illustration of an assembled and reconstructed 3D myocardium image, with uniform sampling of the myocardium from apex to base;

FIG. 8a shows a schematic representation of an opened up myocardium image superimposed with 17 segments of BEV representation;

FIG. 8b shows a schematic representation of an opened up myocardium with extended portions on both the sides by a predetermined fraction;

FIGS. 9a, 9b and 9c show schematic representations of computation and display of average fibrosis, maximum fibrosis in a plane and fibrosis in one r plane in the reconstructed 3D image of myocardium and

FIG. 10 shows a schematic representation of an integrated display of a number of different views and cross-sections of the myocardium.

### DETAILED DESCRIPTION

Before the present subject matter is described in further detail, it is to be understood that the subject matter is not limited to the particular embodiments described, as such may, of course, vary. It shall become abundantly clear after reading this specification, that the subject matter may, without departing from the spirit and scope of the subject matter, also be practiced in other than the exemplified embodiments. For example, other embodiments than the exemplified embodiments are possible, which may vary in shape or size. It shall also become clear that the drawings may not be to the scale. In some other examples, the method may vary to include some additional block(s) or may be practiced in the order different than the order of the blocks discussed in this specification. It is also to be understood that the terminology used herein is for the purpose of describing particular embodiments only, and is not intended to be limiting. It must be noted that as used herein, the singular forms “a”, “an”, and “the” include plural referents unless the context clearly dictates otherwise. The present subject matter provides solution to a number of problems, including but not limited to the problems discussed below.

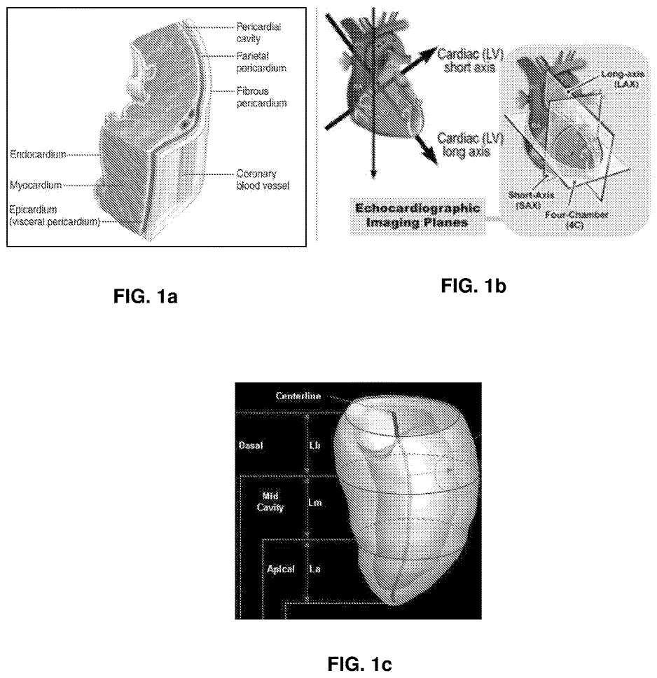

Reference is now made to FIG. 1a. FIG. 1a shows a schematic representation of a cross-section of a myocardium and related constituents of a human heart. The human heart has four chambers namely left ventricle (LV), right ventricle, left atrium and right atrium. The LV is the main chamber that pumps blood to the entire body. Myocardium of LV is specifically studied for heart ailments. Myocardium of LV resembles the shape of a hallow cone. In some applications, the visualization of myocardium is modelled as a hallow cone to study its abnormalities. The heart is supplied with blood by coronary blood vessels (arteries). Ischemia or obstruction to blood circulation in one or more coronary arteries can lead to scarring (dead myocardium tissues) of heart muscle, or fibrosis formation. Fibrosis in myocardium can also occur due to infection, genetic and other causes. Fibrosis caused due to different reasons have different patterns and distributions in the myocardium. One of the main attributes is the extent of transmurality, i.e., presence of fibrosis across a thickness of the myocardium. Thickness of fibrosis may extend from endocardium (inner surface) to epicardium (outer surface).

Reference is now made to FIG. 1b. FIG. 1b shows a schematic diagram of imaging planes and axes of imaging of a human heart. The rectangular coordinate system shown in FIG. 1b is a standard model for visualization and interpretation of heart disorders in a reconstructed image obtained from a diagnostic imaging system. Long axis and short axis are longitudinal and lateral axes as shown in the figure. Images acquired in a plane perpendicular to long axis are called short axis (SAX) image. Images acquired in a plane perpendicular to short axis are called long axis (LAX) images. The orientation of LAX plane may be chosen so as to be able to see all the 2, 3 and 4chamber of the heart, which are called LAX 2C, LAX 3C, LAX 4C respectively. The coordinate system shown in FIG. 1b is found to be useful for the study of Myocardium of LV. The origin is not defined for heart imaging but it is considered to be lying on the long axis and at the centre of the mitral valve.

A 3D volume of left ventricle is made of a set of 2D SAX images

(SAXₖ, for k=0 to z-1) where k is the slice position (i.e., plane) along the cardiac long axis. The ‘z’ SAX images, each taken at a different slice position, makes the entire LV mass and volume. The image may extend beyond the LV and heart. The value of z is in the range of 8-15, with 9 being a typical value. For each imaging plane (i.e., value of k), one can also acquire multiple images (SAXₖ^{RR}) at the same plane but may be acquired at a regular plane and at a fraction of the time intervals between heart-beats. Each one of these SAXₖ^{RR} images capture the beating heart in a different beating cycle (phase). A set of time varying or temporal sequence of images can be captured by a diagnostic imaging system which when played in a rapid sequence can render the movement of a beating heart. Usually SAXₖ^{RR} may consist of 25 images typically covering one full beating cycle of heart from end-diastole to end-systole. End-diastole is the state at which a heart is relaxing and at rest with maximum size. End-systole is the state at which the heart has shrunk to its smallest size and is at rest. A heart moving from diastole to systole position leads to pumping of blood which may be studied in detail by heart specialists for detecting abnormalities of the heart.

Reference is now made to FIG. 1c. FIG. 1c shows a schematic a representation of three sections perpendicular to the long axis of a human heart. The long axis may substantially coincide with a centerline shown in the FIG. 1c. For regional analysis of Left Ventricle (LV) function, the LV is divided into three sections perpendicular to the long axis of the heart as shown. They are called basal, mid-cavity and apical, sectioned across the short axis of the LV. The basal section is designated as the area extending from the mitral annulus to the tips of the papillary muscles at the end diastole. The mid-cavity section is the region that includes the entire length of the papillary muscles. The apical section is the area beyond the papillary muscles to just before where the cavity ends. The true apex or apical cap is the area of myocardium beyond the end of the left ventricular cavity. Each basal, mid-cavity and apical section consists of multiple SAX images.

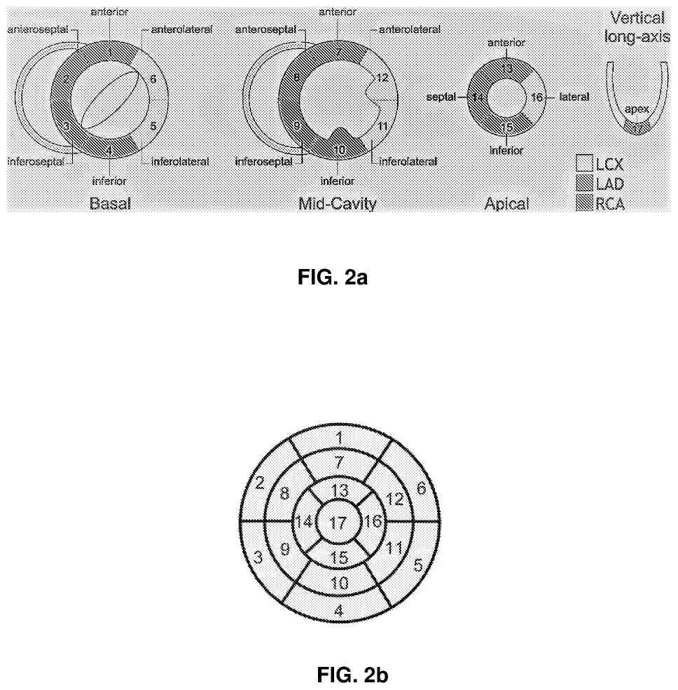

Reference is now made to FIG. 2a. FIG. 2a shows a schematic representation of further sub-division of the three sections shown in FIG. 1c into 17 segments of myocardium. The three sections namely the basal, mid-cavity and the apical sections are shown in FIG. 2a. The basal and mid-cavity sections are further divided into six segments each, as shown in the figure here. The apical segment is divided into four segments and the apex is considered as one segment. Together they constitute 17 segments, of LV myocardium, each approximately representing 1/17 part by volume of the myocardium. This 17 segments map of LV is also called Bull's Eye View (BEV) map.

Reference is now made to FIG. 2b. FIG. 2b shows a representation of a Bull's Eye View (BEV) map of representation of the 17 segments of myocardium. All the 17 segments are concentrated in one graphical representation. This is a standard representation suggested by Physicians of American Heart Association and followed as a standard representation of LV by significant number of Physicians worldwide. The BEV is a standard method of representation of fibrosis percentage in each of the 17 segments, arranged in a circular order, as shown in FIG. 2b. In this graph, it can be seen that the outer-most ring represents for basal segments, the second ring represents mid-cavity segments, the third ring counted from the outer-most ring represents apical segments and the innermost circle represents an apex of a myocardium.

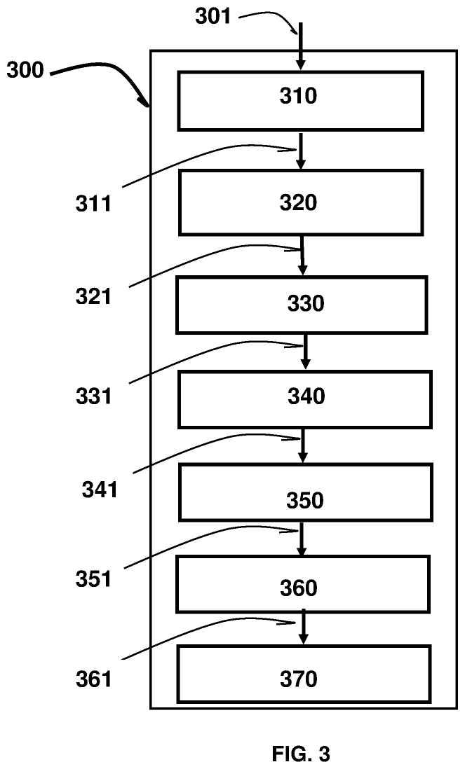

Reference is now made to FIG. 3. FIG. 3 shows a schematic diagram of a method 300 according one embodiment of the present subject matter. The proposed method 300 is shown wherein, steps of the method 300 are shown in blocks 310, 320, 330,340, 350, 360 and 370. Blocks and steps of the method 300 may be interchangeably used as each block is representing a step of the method 300. The inputs for the blocks 310, 320, 330, 340,350, 360 and 370 of the method 300 are shown as 301, 311, 321, 331, 341, 351 and 361respectively. According to the proposed embodiment of the method 300, a first set of volumetric image data 301 comprising cross-sectional images of a myocardium are received at block 310. By volumetric image data, it is meant a set of 2D SAX images (SAXₖ, for k=0 to z-1) where k is the slice position (i.e., plane) along the cardiac long axis. This set of images completely represent a 3D volume of a left ventricle. The ‘z’ SAX images, each taken at a different slice position, makes the entire LV mass and volume.

As a result of the steps of the proposed method 300, as second set of volumetric image data 361 of the myocardium walls of a LV are transformed for r better interpretation and visualization of the myocardium for detecting and estimating the extent of abnormalities such as fibrosis of myocardium walls.

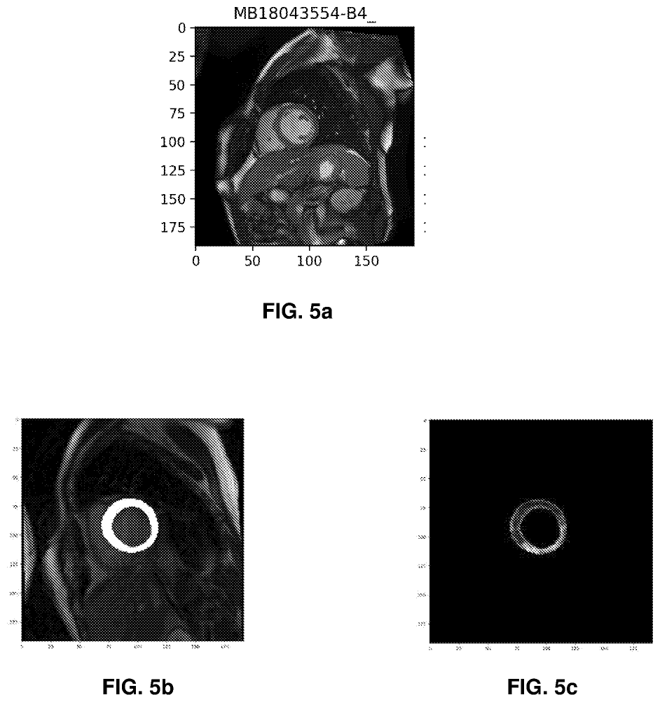

In some examples the pre-processing step 310 of the method may be configured to locate and segment myocardium boundary by one of the well known and standard techniques. Preprocessing a set of cross sectional images of myocardium involves segmenting the shape of myocardium represented in the form of concentric circles by using any one of the standard techniques (Reference: Olivier Bernard et al, “Deep Learning Techniques for Automatic MRI Cardiac Multi-structures Segmentation and Diagnosis: Is the Problem Solved?”, IEEE TAN Journal.) As a result of this preprocessing, centroids of the myocardium and radii of the inner and outer boundaries representing the substantially concentric circles are also obtained after segmentation. The preprocessing step 310 is further explained and illustrated with the help of input and output images in the description of FIG. 5.

In one embodiment, the set of images 301 provided as input to the step 310 may already contain the segmented output of the myocardium images. By segmented output of the myocardium, it means and refers to a set of cross sectional images of myocardium with outer and inner walls of the myocardium segmented with boundaries clearly marked and the coordinates of the boundaries and the pixel values within the inner and outer boundaries can be read and stored. A centroid of each one of the segmented output of the myocardium is computed and made available for further computations. In addition, it is also understood that the outer and inner radii of the boundaries of myocardium are computed and are available for further computations. In such cases, the step 310 may be skipped or by-passed and the segmented set of myocardium images 311 may be directly made available to the step 320 of the method 300. An example of an image comprising a cross sectional image of a myocardium is illustrated and explained in the description of FIG. 5c.

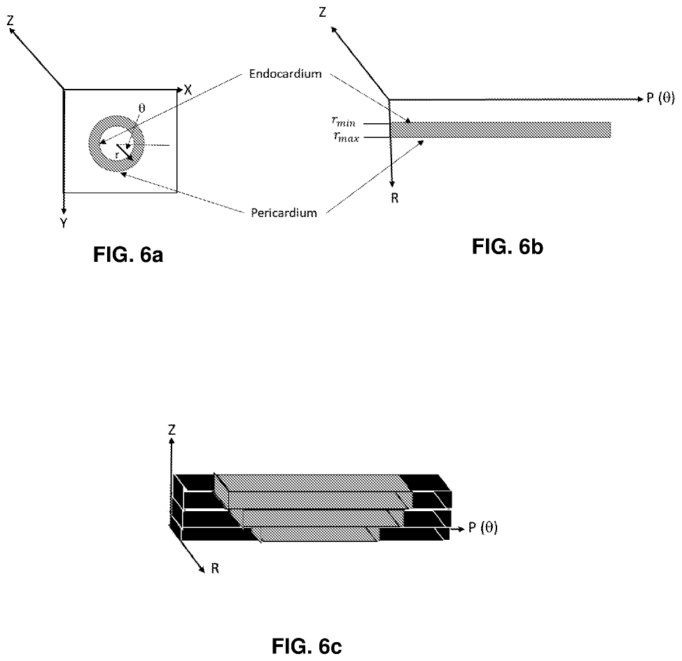

In step 320, each one of the set of segmented images of myocardium is transformed from a polar coordinate system to a rectangular coordinate system. In a segmented myocardium image, a cross section of myocardium is seen as an annulus bounded by the two concentric circles corresponding to the inner and outer walls of a myocardium. The shape of annulus can be represented using cylindrical coordinates or polar coordinates by means of two variables namely an angle (θᵢ) and a radius (rᵢ). By first placing the pixel values in the polar coordinates of discrete values of angle (θᵢ) and radius (rᵢ) on a digital grid, the discrete values can then be transformed in to rectangular (Cartesian) coordinates to obtain a rectangular representation of the annulus shape of the myocardium. The annulus or ring shaped walls of the myocardium as a result of the transformation appears as a rectangular strip or ribbon-like two-dimensional surface. This transformation is done by preserving pixel sizes in different SAX imaging planes, thereby maintaining the aspect ratio of the left ventricle myocardium. Therefore the original pixel values and their locations undergo a transformation and they have to be recalculated to fit into the rectangular grid of discrete values e.g. in (Xᵢ, Yᵢ) coordinates. In the recalculation, the original pixel values are re-sampled to fit the rectangular grid of discrete values in (Xᵢ, Yᵢ). In this manner, a curved plane is reconstructed into a rectangular plane. An example of the transformation step 320 is illustrated and explained in the description of FIG. 6.

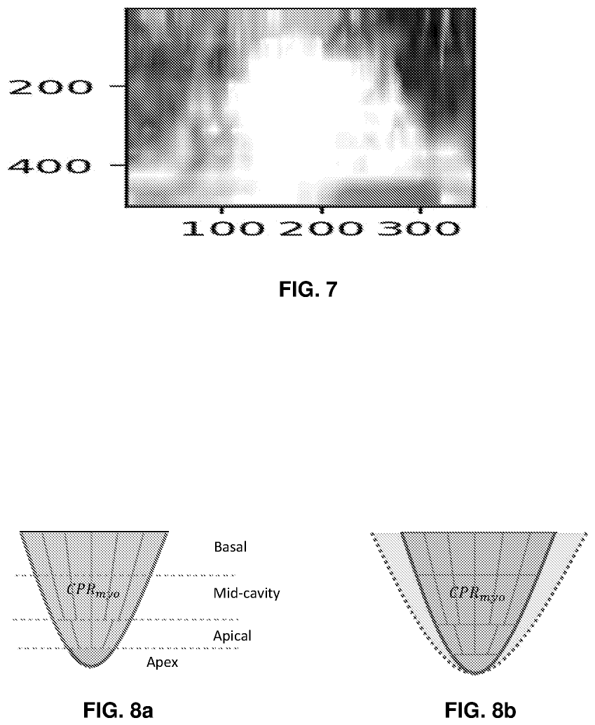

In step 330, a combining operation is performed to combine and assemble all the transformed myocardium images of the slices of myocardium to build a single 3D image of the myocardium for effectively visualizing and studying fibrosis of myocardium tissues. As a result of the combining step 330, a reconstructed set of myocardium images is obtained. The step 330 effectively combines three operations such as (a) stacking up the individual slices in a pre-defined sequence, (b) displacing each individual slice to coincide the edges with an axis and (c) adjusting the scales of different sizes of myocardium walls in order to obtain a combined 3D model of the myocardium and in the shape of a uniform rectangle. In the displacing operation, positions of each rectangular image are adjusted to coincide with one another so that assembling them as a stack is achieved such that adjacent pixels preserve their neighborhood criteria. An example of the combining, stacking up and scaling operations is illustrated and explained in the description of FIG. 7.

A 3D model of a myocardium is reconstructed and displayed by using the proposed method. The proposed method provides a complete 360° view of myocardium from apex to basal and endocardium to epicardium. In this 3D image of myocardium (as illustrated in FIG. 7), the pixel size and aspect ratio are maintained across different SAX imaging planes. In this method of reconstruction of the myocardium, abnormalities such as fibrosis may be detected by a simple image processing operation such as thresholding. The image is adjusted in scale for different sizes of myocardium in different slices. Thus, the proposed method provides a transformation of a set of input SAX images into a 3D volume image of a myocardium thereby making it amenable for further processing using standard image processing techniques and also for complete visualization of myocardium for clinical decision making.

In step 340, the output of the combining step 330 namely the reconstructed set of myocardium images is demarcated with the standard Bull's Eye View (BEV) map. BEV comprises 17 segments of a myocardium walls a standard set for further interpretation. However, in some user groups, 16 or 18 segments may also be in practice as a standard BEV map. Hence there is no limit or constrain in the number of segments used. The superimposition and segmenting of the reconstructed image is further explained with the help of illustrative figures FIG. 8a and FIG. 8b.

In step 350, a set of parameters indicative of the extent of fibrosis of the myocardium are estimated from the respective pixel values of each one of the demarcated plurality of BEV image segments. The fibrosis pixels are defined as those that fall outside a known range of values that fall within the myocardium. The mean (M) and standard deviation (SD) are calculated for all pixels within the myocardium. Pixels having values greater than (M+(n*SD)) (where ‘n’ can take values 2, 3, or 4, . . . and is user-selectable) are considered as fibrosis pixels. From the respective image segments and their respective pixel values, computation of average fibrosis, maximum fibrosis, wall thickness and wall displacement can be configured.

In step 360, superimposing the estimated set of parameters on the respective myocardium segments enables the practitioners to get a better picture of the extent of fibrosis. In the proposed method, the volumetric image and the myocardium segments are adapted to a new method of presentation by modifying the BEV map and superimposing on a reconstructed 3D plane. A visual image and measured values of the abnormality are combined and integrated in the visualization, which was not available in the conventional method of visualization with the BEV map. Estimated sets of parameters 351 indicative of extent of fibrosis of the myocardium at their respective positions on the plurality of BEV image segments to obtain the second set of volumetric image data 361 of the myocardium. In this manner, a better method of quantification and visualization of abnormal tissues of myocardium is provided in the proposed solution.

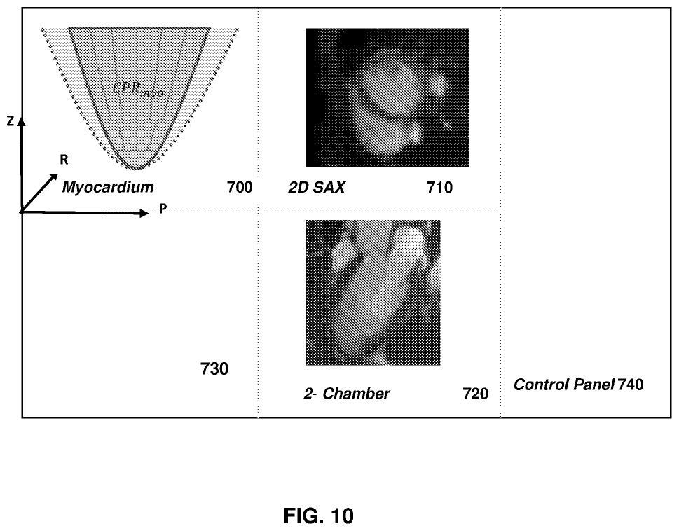

In step 370, the second set of volumetric image data 361 is displayed on a display device. The step370 is further explained in the description of FIG. 10.

In the proposed method 300, a set of myocardium images, whose shape originally resembles a hollow cone, is transformed into a rectangular 3D volume image from apex to base that contains details of myocardium tissues at different slice position and along the respective perimeters of radial positions, starting from endocardium to epicardium. This transformation (a) makes it more amenable to detect pattern in continuum of 3D space of myocardium for better quantification of heart abnormalities; (b) enables a user to see myocardium tissues between endocardium and epicardium and (c) allows better visualization of abnormal myocardium tissues, such as fibrosis.

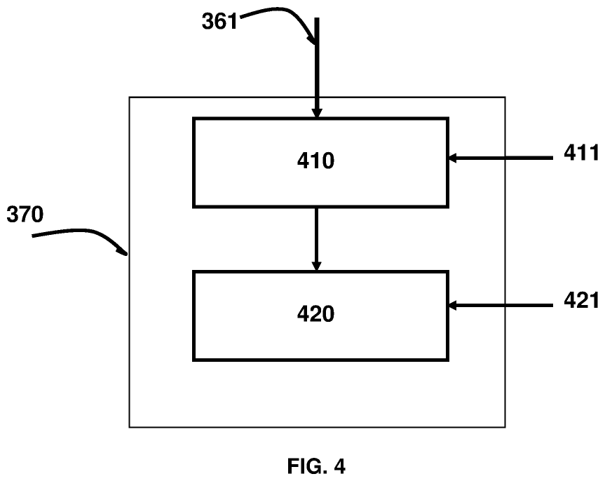

Reference is now made to FIG. 4. FIG. 4 shows a schematic diagram of a step 370 of the method 300 according one embodiment of the present subject matter. The step of displaying 370 comprises two sub-steps 410 and 420 as shown in FIG. 4. The step 410 receives the second set of volumetric image data 361 of the myocardium. The sub-step receives an additional input 411 and the sub-step 420 received an additional step 421. In the step of displaying 370, a plane or slice of the short axis image from the set of short axis images is configured to be displayed based on the user input 411 in the form of a line drawn by the user on the second set of volumetric image data 361. When the user defined line is shifted in position by the user, a new user input is provided as the shift in position 421 and a corresponding slice or plane image is chosen from the set of short axis images and updated on the display in sub-step 420. Therefore, a continuous update is possible when the user draws a line and shifts the line and corresponding cross sectional image from the set of SAX images can be displayed.

Reference is now made to FIG. 5a. FIG. 5a shows a representation of an actual slice of cross-sectional image of a myocardium in a MRI image. The myocardium resembles a shape of a doughnut with a thin-ring like structure at the outer periphery. The ring like structure has an inner boundary known as endocardium and an outer boundary known as epicardium. A set of such cross sectional images of myocardium from apex to base, can be received for reconstruction and display for further study of the myocardium. In one embodiment, an image may be represented as I (x_{N}, y_{N}) of size N×N pixels. It may be noted that the size of pixels in X and Y direction need not be same. In one embodiment, a set of such cross sectional images of a myocardium may be provided as input for the proposed reconstruction method of the myocardium image. In one example, the set of such cross-sectional images of a myocardium may be generated from one of the many medical diagnostic modalities and systems such as Magnetic Resonance Imaging (MRI), Computed Tomography (CT), Ultrasound imaging and Single Photon Emission Computer Tomography (SPECT). Ultrasound imaging systems are also useful in studying the heart abnormalities and conditions. The set of cross sectional images may be made available from any other medical diagnostic modalities.

Reference is now made to FIG. 5b. FIG. 5b shows a representation of a cross-sectional image of the myocardium wherein a detection mask is superimposed on the cross sectional image of the myocardium. The detection mask may be shown to encompass the entire cross section of a myocardium in each one of the set of cross sectional images as a result of a selection of pre-processing methods. There are many such standard techniques available for segmentation and circular object detection, e.g., Hough Transform, Deep Learning (DL) methods and many digital image processing (DIP) methods. Recent methods involving Artificial Intelligence and Machine Learning techniques may be applied to segment such concentric circles representing the inner and outer boundaries of myocardium. (Reference: Olivier Bernard et al, “Deep Learning Techniques for Automatic MRI Cardiac Multi-structures Segmentation and Diagnosis: Is the Problem Solved?”, IEEE TMI Journal) The input images, in one embodiment may contain cross sectional images of myocardium clearly highlighting the wall thickness and inner and outer boundaries by marking the boundary pixels.

Reference is now made to FIG. 5c. FIG. 5c shows the segmented part of the cross sectional view of the myocardium. In the segmented view, pixels belonging only to the myocardium are retained in the slice image of myocardium. In addition, the outer and the inner boundaries substantially forming a shape of concentric circles is highlighted. In the annulus shape of the region of interest, the pixels within the inner and outer walls are taken up from each layer of image i.e., slice position z for further processing and visualization as a 3D reconstructed image of myocardium. In each slide of the image, the shape of myocardium appears as a thin-ring like structure at the periphery of left ventricle.

Reference is now made to FIG. 6a and FIG. 6b. FIG. 6ashows a schematic representation of myocardium in an image for a given slice position z. FIG. 6b shows are representation of the transformed myocardium image for the slice position z. A single layer or slice of the myocardium image is shown in FIG. 6a with three axes ‘X’, ‘Y’ and ‘Z’ for better understanding of the transformation that is implemented in the proposed method of reconstruction of myocardium from the multiple such layers or slices of images obtained from an image capturing system. The axes X and Y form a plane on which the single layer image is placed. After the transformation, a concentric rings like structure shown in FIG. 6a is transformed from polar coordinates with radius r and angle θ into a strip or a ribbon like structure in FIG. 6b to be represented in a rectangular coordinate system having ‘R’, ‘P’ and ‘Z’ axes. The radius r varies from rₘᵢₙ to rₘₐₓ as represented on the R axis. In FIG. 6b, rₘᵢₙ value which corresponds to the endocardium is placed closer to the ‘P’ axis compared to rₘₐₓ that corresponds to the epicardium. In another embodiment, rₘₐₓ can be positioned closer to the P axis and vice versa. This transformation process is repeated for each and every slice or layer of the set of myocardium images, thereby obtaining a rectangular image of each slice or layer.

Reference is now made to FIG. 6cshows an illustration of assembling of three transformed myocardium images obtained from three slices of the myocardium images. FIG. 6cshows a representation of three image slices assembled in a three dimensional frame. The coordinate system and the axes shown in FIG. 6b are rotated by 90° in comparison to the view shown in FIG. 6b to present a proper view of the stacked up image slices in 3D. FIG. 6b illustrates the view obtained after the proposed transformation and stacking up of the individual images obtained from the slices of cross sectional images of myocardium resulting in a unique way of visualizing the myocardium.

As a first step of image pixel values at each discrete locations of angle (θᵢ) and radius (rᵢ) are computed. Since there is a pair of circles in each segmented myocardium image, the radius of the shape varied from a minimum value to a maximum value viz., rₘₐₓ and rₘᵢₙ. It can be understood by a skilled person that as the value of radius (rᵢ) varies from rₘₐₓ to rₘᵢₙ, pixel widths along the radius (rᵢ) may vary proportionately to their distance from the origin. Therefore, pixel widths have to be computed for each discrete value of angle (θᵢ) ranging from rₘₐₓ to rₘᵢₙ in a finite number of steps. A set of calculations for pixel values are computed using an equation with a set of variables. From the polar grid, a set of coordinates of rectangular grid are computed in the next step. This is a straight forward computation obeying the equations X=r Sin (θ) and Y=r Cos (θ). A second set of image pixels corresponding to the set of discrete rectangular coordinates are computed. Subsequently, corresponding pixel values are computed to fit into the rectangular grid. In this manner, a curved surface is converted to a rectangular plane. A complete set of computations for the curved surface reconstruction to a rectangular plane is shown in Appendix ‘A’ titled “Methodology for Myocardium reconstruction”.

Reference is now made to FIG. 7. FIG. 7 shows an illustration of an assembled and reconstructed 3D myocardium image, with uniform sampling of myocardium from apex to base. FIG. 7 shows an illustration of an assembled and reconstructed 3D myocardium image after adjusting it for different sizes of the myocardium in different slices.

In FIG. 7, Y-axis represents layers or slices of images or slice positions. In FIG. 7, the X-axis represents angular position of points along the perimeter of a circle centered at the center of a myocardium. The angular value varies from 0 to 360°, measured with respect to horizontal (X) axis. Hence the proposed method of reconstruction of the myocardium image provides a complete 360° view of myocardium from apex to basal and also from endocardium to epicardium. This makes it very easy to apply image processing techniques, such as thresholding, to segment myocardium for characterizing abnormal structures such as fibrosis e.g., the white patch seen in FIG. 7.

Reference is now made to FIG. 8a. FIG. 8a shows a schematic representation of an opened up myocardium image superimposed with 17 segments of the BEV representation. Effectively, a 3D conical shape of the myocardium is sliced in the vertical plane and spread sidewise to obtain a clear view of the walls. The image is labeled as CPR_{myo} and the three sections namely the basal, the mid-cavity and the apical sections are shown in FIG. 8a.

Reference is now made to FIG. 8b. FIG. 8b shows a schematic representation of an opened up myocardium with extended portions on both the sides by a predetermined fraction. In general, it is observed that a 60° extension may result in a better representation. The extension is created by reproducing the pixels from the opposite edge. Abrupt cutting off of the edges is avoided. For example, a fraction of the right side of the myocardium wall is copied and pasted on the left side and vice-versa to generate a myocardium image with a wrap around. One of the advantages of the presentation in a wrap around method is to preserve the adjacency of pixels thereby enabling the study of continuity of fibrosis.

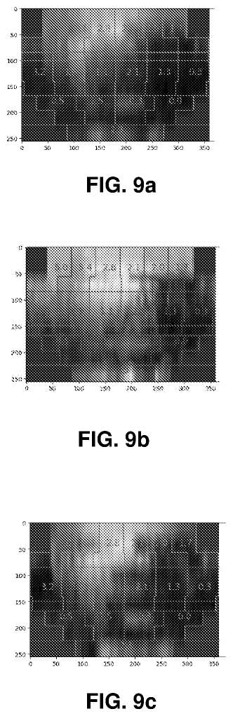

Reference is now made to FIGS. 9a, 9b and 9c. FIGS. 9a, 9b and 9c show schematic representations of computation and display of average fibrosis, maximum fibrosis in a plane and fibrosis in one r plane in the reconstructed 3D image of myocardium. From the reconstructed image as illustrated in the description of FIG. 7, an extent of fibrosis can be calculated and characterized by means of pixel values. Average fibrosis is computed in all the segments of the reconstructed image and the computed parameter values are superimposed on the respective segments for better interpretation of the abnormality. Average fibrosis in all the CPRᵣ_{ᵢ}^{R} images (for i=0 to R-1), as well as the maximum value in any of the plane are computed and displayed on FIG. 9a and FIG. 9b respectively. It can be seen that there are four sections of myocardium corresponding to basal, mid-cavity, apical and apex, separated by horizontal green lines. The 17 image segments corresponding to a BEV map can be seen on the myocardium, demarcated by red lines. Hence possibilities of unique perspective, visualization and interpretation are available for the first time to Physicians wherein the fibrosis values in each of the seventeen segments of myocardium are superimposed on the adapted BEV map, while maintaining special relationship and neighbourhood information of each of the segments. A skilled person can apply this proposed solution and achieve significant improvement over the conventional BEV presentation map of visualizing myocardium of human heart. The proposed solution addresses many of the shortcomings of the BEV representation map of myocardium of human heart.

Reference is now made to FIG. 10. FIG. 10 shows a schematic representation of an integrated display of different views and cross-sections of the myocardium. In one embodiment, the proposed method can be applied for an integrated visualization of the second set of volumetric image data of the myocardium in tandem with a plurality of related set image data of human heart.

In another embodiment, the display area is divided in to five display areas as illustrated in FIG. 10. In this illustration, a control panel 740 is configured in one of the five display areas. The remaining four of display areas 700, 710 and 720 and 730 are configured to display various types of images such as 3-dimensional and two dimensional images, black & white and colour images, graphical and alphanumeric images. The display parts may be provided with independent controls of basic image quality adjustments such as hue, saturation, contrast, brightness etc. In one of the embodiments, display area 700 is configured to display the second set of volumetric image data 371 wherein the reconstructed myocardium image superimposed with the demarcations of BEV image segments and the computed parameters indicative of extent of fibrosis of the myocardium. In one embodiment, by means of a mouse interface or by touching a touch sensitive display surface, a set of parameters of fibrosis may be configured to be displayed on an individual or a plurality of BEV mapped segments.

In another embodiment, the display area 710 is configured to display a 2D-SAX image. In yet another embodiment, the display area 720 is configured to display a 2-Chamber image. In another embodiment, the display area 730may be configured to display a 4-Chamber image. In yet another embodiment, the display areas 730 may be configured to display a volume image of myocardium with a colour overlay for displaying different colour codes indicating the extent of fibrosis of myocardium tissues may be displayed.

In one embodiment, the display area 700 is configured to receive and display a horizontal line input from a mouse interface or a keyboard interface or any such interface. The horizontal line intersects the displayed second set of volumetric image data 371 at a specified level and it may be configured to select a particular slice position (z value) of the Z-axis shown in display area 710. A corresponding 2D-SAX layer or slice (short axis view) may be displayed in the display area 710. By dragging the horizontal line up/down through a user interface, the display may be configured to select a different slice position of the corresponding 2D-SAX layer or slice image.

In one embodiment, any one of the display area may be configured to display a time series images for the given slice position as a movie sequence, thereby representing wall motion while the heart is beating. This can be done by clicking on the play button in the navigation section of control panel740.

The reconstructed image of the myocardium can be subjected to further processing in order to obtain segmented volumes of fibrosis from the reconstructed image of the myocardium. The segmentation can range from a simple thresholding based on the gray values of the reconstructed myocardium images or advanced digital image processing techniques based on human perception or artificial intelligence methods. From the segmented volume images, the abnormal tissues can be isolated and studied. An accurate depiction of the dimensions and extent of fibrosis may be obtained to study the extent of trans-murality of the fibrosis and further actions such as interventions or prognosis of the disorder can be ascertained.

The subject matter further provides a device configured to perform processing of a first set of volumetric image data 301 comprising cross-sectional images of a myocardium comprising pixel values and displaying a second set of volumetric image data 361 of the myocardium, the device comprising a processing element adapted to carry out the steps of the method 300.

The steps of pre-processing 310, pre-processing 320, transforming 330, combining 340, demarcating 350, superimposing 360, and displaying 370 the second set of volumetric image data 361 of the myocardium on a display device can be implemented using one or more processors or using some form of hardware or firmware with embedded controllers such as microcontrollers. The processing power of the display may be enhanced by additional graphics accelerators configured for dedicated display functions. A combination of hardware and software in varying degrees and proportions may be implemented to achieve the proposed solution.

### Appendix A: Methodology for Myocardium Reconstruction

```
START{
    Input: A 3D volume image SAX= U {SAX_{z}, z ∈[0,1,2,...z-1] }with size (N × N ×
    z ) where each SAX_{z} image for slice position z is a N × N(pixels) image.
    1. Δθ, increment of ‘θ’ along the perimeter, is set to a constant;
    2. For each 2D image SAX_{z}, For z ranging from 0 to z-1, do the following :
      a. Multiply each SAX_{z} image with respective myocardium masks M_{z} to
        form respective image lm_{z}.
      b. Calculate centre location(Cₓ^{z},C_{y}^{z})of the myocardium mask.
      c. Calculate minimum radius rₘᵢₙ^{z} and maximum radius rₘₐₓ^{z} from the
        centre of mayocardium at different slice positions (such that for r ≥
        rₘᵢₙ^{z} and r ≤ rₘₐₓ^{z} there is at least one myocardium pixel in the mask
        M_{z}
      d. CPR_{2D}^{z} = Compute CPR (lm_{z}, r^{min}_{z}, rₘₐₓ^{z}, Δθ_{z})
    3. CPR_{3D}^{z} = U { CPR_{2D}^{z}, for z ∈ [0,1,2,...z-1] }
    4. Return CPR_{3D}^{z}, a 3D image with axes as perimeter, radius and slice
      position with size R × P × z
    Here, R = (max)_{z} (rₘₐₓ^{z} − rₘᵢₙ^{z}) + 1;
          P = 2π/Δθ where Δθ is a constant angular increment;
          and z = number of SAX slice images
      CPR_{2D}^{z} = CPR_{3D}^{z}[:, :, z] is the z^{th} projection and
      CPR_{2D}ⁱ = CPR_{3D}[i, :, :] represents the complete myocardium (apex to
    base) for a particular radial position i.
    Methodology to transform lm_{z} to an image CPR_{2D}^{z}of size R × P with axes as
    radius rand perimeter p.
    Compute CPR( lm, rₘᵢₙ, rₘₐₓ, Δθ):
      a. For θ ranging from 0 to 2π with incrementΔθ
      b. For r ranging from rₘᵢₙ to rₘₐₓ
        i. Compute(x, y) coordinates of the point(r, θ) from the centroid
        (Cₓ, C_{y}) of lm
        ii. Proj_{2D} (r, θ) = lm(x,y)
    2. Return Proj_{2D} (It can also be called as CPR_{2D}^{z})
  } END
```

A further embodiment of the methods may be implemented in a set of computer implementable instructions and executed with a combination of hardware involving a processor, an internal or external memory and the said computer implementable instructions for the processor that may be stored in the internal or external memory. In a further embodiment, the method may also be implemented in a hardware, firmware, programmable logic gates. In a further embodiment, the method may be implemented in a conventional computer with suitable interfaces and computer implementable instructions. In this manner, a system can be built in software or hardware or as a combination of hardware and software.

In a further embodiment, with the advent of internet and interconnectivity of computers, it may be possible and convenient to stored large amounts of data in a server located anywhere in the internet ‘cloud’ and download the desired data by having a suitable internet connectivity, using a conventional client-server architecture. In a further embodiment, the hardware for downloading the battery profile may be accordingly configured as a client.

In a further embodiment, Internet of Things (IoT) facilitate interconnectivity of various devices such as sensors, mobile devices, automobiles, data concentrators etc., and interchange of data and control over the internet.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” or “one or more of the following of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

In addition, the use of “adapted to” or “configured to” in this disclosure is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

All examples and conditional language recited in the present disclosure are intended for pedagogical objects to aid the reader in understanding the present disclosure and the concepts contributed by the inventor to furthering the art, and are to be construed as being without limitation to such specifically recited examples and conditions. Although embodiments of the present disclosure have been described in detail, various changes, substitutions, and alterations could be made hereto without departing from the spirit and scope of the present disclosure.

Elements of different embodiments described herein may be combined to form other embodiments not specifically set forth above. Various elements, which are described in the context of a single embodiment, may also be provided separately or in any suitable sub-combination. Other embodiments not specifically described herein are also within the scope of the following claims.

What is claimed is:

1. A method(300) for processing a first set of volumetric image data(301) comprising cross-sectional images of a myocardium and displaying a second set of volumetric image data(361) of the myocardium, the method comprising:
pre-processing(310) the first set of volumetric image data(301) received from an image capturing system to obtain a segmented set of myocardium images(311) and their respective centroids and radii;
transforming(320) each one of the segmented myocardium images from a polar coordinate system into a rectangular coordinate system using their respective centroids and radii to obtain a transformed set of myocardium images(321);
combining(330) the transformed set of myocardium images to obtain a reconstructed set of myocardium images(331);
demarcating(340) a plurality of image segments conforming to a bull's eye view (BEV) map on the reconstructed set of myocardium images(341);
estimating(350) a set of parameters indicative of extent of fibrosis of the myocardium from the respective pixel values of each one of the demarcated plurality of BEV image segments;
superimposing(360) the estimated sets of parameters(351) indicative of extent of fibrosis of the myocardium at their respective positions on the plurality of BEV image segments to obtain the second set of volumetric image data(361) of the myocardium and
displaying(370) the second set of volumetric image data(361) of the myocardium on a display device.

2. The method of claim 1, wherein the estimated set of parameters(351) indicative of the extent of fibrosis are selected from a group consisting essentially of average fibrosis, maximum fibrosis, wall thickness and wall displacement of the myocardium.

3. The method of claim 2, wherein the method (300) further comprises a step of navigating in radial direction to display respective cross-sectional views as a 2-dimensional image derived from the second set of volumetric image data(361) of the myocardium.

4. The method of claim 3, wherein at least one of the estimated set of parameters is displayed on each one of the derived 2-dimensional image of the second set of volumetric image data (361) of the myocardium.

5. The method of claim 1, wherein the first volumetric image of the myocardium is received from at least one of the CT, MRI, ultrasound, X-ray, PET, and SPECT system representations of the myocardium.

6. The method of claim 1, wherein the method further comprises steps of
attaching a pre-decided percentage from the right side of the reconstructed set of myocardium images to the left side of the reconstructed set of myocardium images; and
attaching a pre-decided percentage from the left side of the reconstructed set of myocardium images to the right side of the reconstructed set of myocardium images,
before demarcating the plurality of image segments conforming to the bull's eye view (BEV) map, thereby providing an augmented view of the reconstructed set of myocardium images.

7. The method of claim 1, wherein the step (370) of displaying further comprises receiving and displaying at least one of: a short axis image set, a 2-Chamber image set, a 3-Chamber image set and a 4-Chamber image set from the image capturing system in a plurality of display windows in addition to the second set of volumetric image data(361) of the myocardium.

8. The methods of claim 7, wherein step (370) of displaying further comprises displaying at least one of: the short axis image set, the 2-Chamber image set, a 3-Chamber image set and a 4-Chamber image set as a sequence of a plurality of images in a predetermined time sequence, thereby displaying the beating of a heart in the display device.

9. The method of claim 1, wherein the step(370) of displaying further comprises:
displaying a plane (410) of the short axis image set corresponding to a user defined line (411) on the second set of volumetric image data(361) and
updating the displayed image(420) with the corresponding plane of the short axis image set corresponding to the shifting of the user defined line (421) on the second set of volumetric image data(361).

10. The method of claim 1, wherein the step of displaying further comprises displaying
on a display window at least one of the 2-Chamber image, 3-Chamber image and 4-Chamber image interactively chosen from the respective set of images.

11. A device configured to perform processing of a first set of volumetric image data (301) comprising cross-sectional images of a myocardium comprising pixel values and displaying a second set of volumetric image data(361) of the myocardium, the device comprising a processing element adapted to carry out the steps of:
pre-processing the first set of volumetric image of the myocardium received from an image capturing system to obtain a segmented set of myocardium images and respective centroids and radii;
transforming each one of the segmented myocardium images from a polar coordinate system into a rectangular coordinate system using the respective centroids and radii to obtain a transformed set of myocardium images;
combining the set of transformed set of myocardium images to obtain a set of reconstructed set of myocardium images;
demarcating a plurality of segment boundaries conforming to a bull's eye view (BEV) map on the reconstructed set of myocardium images;
estimating a set of parameters indicative of extent of fibrosis of the myocardium from the respective pixel values of each one of demarcated plurality of BEV image segments;
superimposing the estimated sets of parameters indicative of extent of fibrosis of the myocardium at their respective positions on the plurality of BEV image segments to obtain the second set of volumetric image data image and
displaying the second set of volumetric image data of the myocardium on a display device.
